(19) United States
(12) Patent Application Publication
(10) Pub. No.: US 2011/0022038 A1
(43) Pub. Date: Jan. 27, 2011

(54) Device for Treating Occlusions, and Method for Treating Occluded Vessels

(76) Inventors: Niranjan Seshadri, Sarasota, FL (US); Nagraj Seshadri, Providence, RI (US)

Correspondence Address: PARFOMAK, ANDREW N.;NORRIS MCLAUGHLIN & MARCUS PA, 875 THIRD AVE, 8TH FLOOR, NEW YORK, NY, 10022, US

(21) Appl. No.: 12/867,847

(22) PCT Filed: Mar. 28, 2008
(86) PCT No.: PCT/US2008/058620

§ 371 Date: Oct. 1, 2010

(51) Int. Cl. A61B 17/22; A61F 2/01; A61F 2/82; A61B 18/24

(52) U.S. Cl. 606/7; 606/200; 623/1.11; 606/159

(57) ABSTRACT

The present invention relates to devices and procedures for medical care and pertains in particular to medical devices and techniques for treatment of occlusions, particularly chronic total occlusions in vessels, particularly blood vessels of human or animal subjects.

### FIELD OF INVENTION

This invention relates to devices and procedures for medical care and pertains in particular to medical devices and techniques for treatment of occlusions, particularly chronic total occlusions.

### BACKGROUND OF THE INVENTION

A chronic total occlusion in the human arterial circulation is a total blockage of the arterial circulation from plaque buildup within the lumen of the blood vessel. The plaque may consist of cholesterol and calcium deposits. In general, a chronic total occlusion is one that has been present for three months or longer, in some cases even years. A chronic total occlusion may occur anywhere in the arterial tree, such as the heart or within the peripheral arteries in the arms and legs.

A chronic total occlusion occurring the lower extremity arterial circulation may be clinically relevant as it may cause pain the legs with walking, in more serious cases it may lead to non-healing ulceration or even loss of limb.

A chronic total occlusion occurring in a vessel near the heart can have more devastating consequences such as a stroke, or a heart attack.

Percutaneous treatment options for treating chronic total occlusions commonly involve balloon angioplasty, stent placement and plaque excision. Before these treatment options are carried out, a guidewire (over which devices such as balloons, stents etc. are delivered to the site of blockage) needs to cross the blockage. Frequently the plaque comprising the chronic total occlusion is “rock-hard” and needs a lot of forward pressure with the guidewire. This forward pressure exerted on the guidewire may cause the supporting catheter (through which the guidewire is delivered to the site of blockage) to back-up. Additionally, the guidewire may not enter the center of the plaque, but may enter the wall of the vessel, or even exit the vessel. This may be due to the eccentric orientation of the supporting catheter within the lumen of the artery proximal to the site of occlusion.

Various devices and methods for treating occlusions and occluded vessels have been proposed in the art.

For example, in U.S. Pat. No. 5,263,959 and in U.S. Pat. No. 5,423,846 there is described a dottering auger catheter system designed for penetration of tight stenoses or total occlusions (interchangeably referred to herein also as “blockages”) as a precursor to balloon angioplasty, atherectomy, or any other vessel opening means that requires an initial passageway. Specifically, the dottering auger catheter system consists of a centering catheter and a dottering auger catheter which is a catheter that opens a passageway through an arterial blockage by passing its wedge shaped distal end through that blockage causing outward plastic deformation of the “plaque”; i.e., angioplasty. A centering catheter that surrounds the dottering auger catheter can be used to center the distal end of the auger catheter just proximal to the blockage. The purpose of the auger type of dottering auger catheter is to penetrate through the blockage by means of a self-tapping screw at the catheter's distal end. Once the first thread of the screw is pushed into the blockage, rotation of the catheter resulting from turning a handle at the catheter's proximal end will cause the screw to pull itself through the blockage while a push force on the handle is also applied. Once the auger has dottered a passageway through the blockage, the auger catheter is removed and replaced with a guide wire. Once the guide wire is in place through the newly opened passageway, the centering catheter is removed and conventional balloon angioplasty or atherectomy can be performed to further enlarge the hole through the blockage thus restoring adequate blood flow.

U.S. Pat. No. 5,833,650 describes a catheter assembly for treatment of a stenosis in a lumen in a blood-carrying vessel comprising a first flexible elongate tubular member having proximal and distal extremities. A first inflatable balloon is coaxially mounted on the distal extremity of the first flexible elongate tubular member and has a main lumen and an aspiration lumen exiting through the distal extremity. A second flexible elongate tubular member has proximal and distal extremities and has a second inflatable balloon coaxially mounted on the distal extremity and has a blood perfusion lumen extending therethrough. The second flexible elongate tubular member is slidably mounted in the main lumen of the first flexible elongate tubular member. The first balloon is positioned so that it is adjacent to but proximal of the stenosis and is thereafter inflated to create a first occlusion in the lumen in the vessel. A negative pressure is created in the lumen in the vessel distal of the first balloon through the aspiration lumen. The second balloon is positioned distal of the stenosis and is inflated to create a second occlusion in the lumen of the vessel to form a working space between the first and second balloons which brackets the stenosis. Blood is perfused through the blood perfusion lumen distal of the second occlusion.

U.S. Pat. No. 5,972,019 disclosed a mechanical clot treatment device which may be delivered through an intravascular catheter. The device includes has a core element, typically a core wire, placed around the distal end of which is a collapsible and preferably self expanding cage assembly. The proximal end of the cage is typically is affixed to an actuator in such a way as to allow expansion of the cage after deployment. The cage assembly may have a generally conical distal or “trailing” portion when expanded and also a proximal section. The cage assembly may be used for collecting emboli or for displacing them to allow blood flow to resume. The proximal section may have various uses, e.g., for centering the collector assembly in the vascular lumen or for gathering larger amounts of the targeted clot or to act as a passageway for fluid flow. The assembly further may have an actuator which permits or causes the collector assembly to expand after deployment.

U.S. Pat. No. 6,391,044 discloses a removable vascular filter system for blocking micro- and macro-emboli while allowing the continued perfusion of blood comprises a filter membrane positioned on a guidewire, wherein a free end of the membrane sits tightly against the guidewire when the filter membrane is in a collapsed state and wherein the filter has a means for deploying the filter membrane to assume a position substantially normal to the longitudinal axis of the guidewire. The filter membrane is comprised of a fine mesh material which has a pore size capable of blocking emboli while allowing continued blood flow, a preferred embodiment of which comprises regularly spaced, laser-formed holes

Further devices known to the art which are presently commercially available include the LUMEND FRONTRUNNER (ex. Cordis corp.). This device uses a reverse scissor spreading-type action, also referred to as blunt microdissection, to separate the plaque. However this device is not without certain drawbacks including, the inability to enter the blockage primarily with the guidewire, which may increase changes of perforating the arterial wall, which is a potentially devastating complication. Additionally, the device provides no centering mechanism with this device to ensure passage of the tip through the center of the plaque.

A further commercially available device is the ASAHI TORNUS CATHETER (Abbott corp.). This device is a supporting catheter for crossing chronic occlusions and has several hair-thin, stainless steel strands braided together to enhance the strength of the catheter. This device also has certain shortcomings, namely in that the device may undesirably back up with forward pressure on the guidewire.

Therefore, there exists a need for an improved device useful for the treatment of occlusions, particularly chronic total occlusions which addresses and overcomes shortcomings in prior art devices. There also exists a real and urgent need in the art for improved methods for the treatment of occlusions, particularly chronic total occlusions which utilized improved devices.

In one aspect the present invention provides an improved device useful for the treatment of occlusions. The said device comprises an outer catheter and an inner catheter, said inner catheter having disposed on a part of its outer surface a collapsible cage, wherein the said inner catheter and cage are adapted to be slidably deployed from the interior of said outer catheter.

In another aspect the present invention provides an improved device useful for the treatment of occlusions. The said device comprises an outer catheter and an inner catheter, said inner catheter having disposed on a part of its outer surface a collapsible cage, wherein the said inner catheter and cage are adapted to be slidably deployed from the interior of said outer catheter, wherein the device further comprises a control device at or proximate to the end of the device.

In a further aspect of the invention there is provided a process for the treatment of occlusions, particularly chronic total occlusions which process comprises the steps of:
positioning the distal end of a device which comprises an outer catheter and an inner catheter, and a treatment article (i.e., a guide wire, a balloon tipped catheter, a dottering auger, and the like) slidably deployable from the interior of said inner catheter, said inner catheter having disposed on a part of its outer surface a collapsible cage, wherein the said inner catheter and cage are adapted to be slidably deployed from the interior of said outer catheter within a vessel adjacent to the locus of an occlusion, and thereafter,
deploying said collapsible cage from the interior of the outer catheter, and,
optionally but preferably treating said occlusion utilizing the treatment article.

In a still further aspect of the invention provided a process for the treatment of occlusions, particularly chronic total occlusions which process comprises the steps of:
positioning the distal end of a device which comprises an outer catheter and an inner catheter, and a guide wire slidably deployable from the interior of said inner catheter, said inner catheter having disposed on a part of its outer surface a collapsible cage, wherein the said inner catheter and cage are adapted to be slidably deployed from the interior of said outer catheter within a vessel adjacent to the locus of an occlusion, and thereafter,
deploying said collapsible cage from the interior of the outer catheter, and,
deploying said guide wire or other element associated with said guide wire into or across the locus of an occlusion in a vessel in order to treat the occlusion,
collapsing said collapsible cage by slidably deploying said inner catheter into said outer catheter and, removing the inner catheter and outer catheter from the vessel, while retaining the guide wire within the vessel.

These and other objects of the invention will described with reference to the following specification and description of preferred embodiments and from the accompanying figures.

### BRIEF DESCRIPTION OF THE DRAWINGS

FIG. 1 is a perspective view of the distal end of a device for crossing a chronic total occlusion, in accordance with a preferred embodiment of the present invention.

FIG. 1A is a transverse view of a part of the device of FIG. 1.

FIG. 2 is a perspective view of the distal end of the device of FIG. 1, illustrating the inner catheter being retracted in the outer catheter, and the cage being fully collapsed within the interior of the outer catheter.

FIG. 3 is a perspective view of the device of FIGS. 1 and 2, illustrating cage as being partially deployed or opened.

FIG. 4 is a perspective view of the device of FIGS. 1, 2 and 3, illustrating cage as being fully deployed or opened.

FIG. 5 is a plan transverse view of the distal end of the device within the lumen of a blood vessel illustrating cage as being fully deployed or opened and illustrating the centering of the distal end of the device within said blood vessel by virtue of the deployed cage.

FIGS. 6A-6F illustrate in several perspective views various embodiments of the cage according to the present invention.

FIG. 7 depicts a plurality of cross-sectional configurations of the elements which may be used to form the cage.

FIG. 8 depicts a preferred embodiment of the device of the invention, including both proximal and distal ends of the device.

FIG. 9 depicts in detail a control device useful at the proximal end of the device according to the invention.

FIG. 9A depicts in detail a further embodiment of a control device useful at the proximal end of the device according to the invention.

FIG. 9B depicts in detail a further embodiment of a control device useful at the proximal end of the device according to the invention.

FIGS. 10A, 10B and 10C depict the control device according to FIG. 9 in three different modes or operating states.

FIG. 11 depicts a locking means useful in the device according to the invention.

It is believed that the present invention will be better understood from the following description of preferred embodiments, taken in conjunction with the accompanying drawings, in which like reference numerals identify identical elements throughout each of the figures

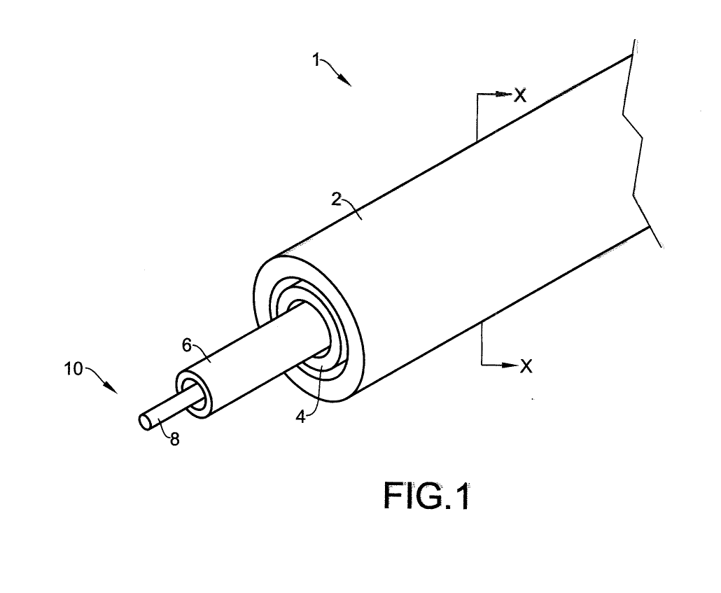

FIG. 1 depicts in a perspective view a portion of the distal end of a device 1 according to a preferred embodiment of the instant invention. As is shown thereon the device 1 comprises an outer catheter 2 encasing an inner catheter 4 in a concentric relationship thereto. Optionally, within the inner catheter is also provided a sleeve 6 (which may be a further catheter or other element, albeit not illustrated) which is depicted as also being concentric with the outer catheter 2 and the inner catheter 4. Contained within this sleeve is a guidewire 8. It is to be understood that the outer catheter 2, inner catheter 4, sleeve 6, and guidewire 8 are all laterally slideable with respect to each other and it is to be further understood that each is independently moveable with respect to any of the other named elements.

For sake of convenient and uniform reference, the distal end 10 of the apparatus 1 will be understood as being that portion of the apparatus which is inserted into the human subject, viz., patient, in need of treatment while the other opposite, proximal end 80 (not shown in FIG. 1) is to be understood as that part of the apparatus 1 which is intended to be extended from the human body and operated by a surgeon, other medical personnel, machine or robot who manipulates the placement of the distal end 10 of the apparatus 1 proximate to an occlusion in a vessel, particularly an arterial vessel within a human or animal patient.

FIG. 1a shows a transverse, cross-sectional view along line X-X of the device 1 illustrating the interior arrangement of the foregoing elements in more detail. As is readily seen therein, the outer catheter 2 completely encloses the inner catheter 4 and is concentric therewith. There is also visible from this depiction an inter-catheter gap 12 which is the space formed between the inner wall 14 of the outer catheter 2, and the outer wall 16 of the inner catheter 4. This gap 12 can be minimal and need only be sufficiently large so as to not unduly impede the movement of the inner catheter 4 within the outer catheter 2 when the two catheters are moved with respect to each other. Ideally, a small gap exists, generally on the order of from about 0.1 millimeters-0.8 millimeters (“mm”), preferably 0.2 mm-0.4 mm in order to provide for a readily smooth motion with minimal friction between the inner catheter 4 and the outer catheter 2. As is also visible from FIG. 1a that the outer catheter 2 is essentially concentric with inner catheter 4 although this is a preferred embodiment, and is not essential to the operation of the invention. That is to say, that although not depicted it is to be understood that different cross-sectional geometries other than concentric circles or cylinders as depicted in FIG. 1a may be presented and used in some or all of the device 1. For example, it is clearly contemplated that while it may be convenient that the outer catheter 2 have a generally circular outer wall 18 such does not foreclose the possibility that a non-circular geometry exists within the device 1 according to the present invention. For example, it is contemplated that the inner catheter 4 may have a shape other than a generally circular configuration i.e. can be polygonal, i.e. hexagon, octagon, decagon, or elliptical or oval which may be advantageously considered, as such reduce the amount of surface of the outer-wall 16 of the inner catheter 4 which may come into interfacial contact with the inner wall 14 of the outer catheter 2. The use of such non-circular outer wall 16 of the inner catheter 4 may potentially reduce to a great extent the interfacial friction between the inner wall 14 of the outer catheter 2 and the outer wall 16 of the inner catheter 4 thereby reducing the amounts of force needed to slide the inner catheter 4 within the outer catheter 2.

As is depicted in the embodiment according to FIG. 1a, the inner catheter 4 further includes within its interior a sleeve 6. The sleeve 6 has an outer wall 20 which is separated by a sleeve gap 22 from the inner wall 24 of the inner catheter. Again, while this gap may be minimal, it is advantageously on the order of about 0.1 mm-0.8 mm, preferably 0.2 mm-0.4 mm. Such gap provides for the more ready movement of the sleeve 6 within the interior of the inner catheter 4. As is shown in the figure, it is seen that in this preferred embodiment that the sleeve 6 is essentially concentric with both the inner catheter 4, and the outer catheter 2.

Again, it is to be understood as has been generally discussed with reference to the relationship between inner catheter 4 and the outer catheter 2 that the outer wall 20 of the sleeve 6 can also have a configuration other than the generally circular configuration as seen on FIG. 1a. That is to say that the outer wall 20 can take a different shape, such as a polygon, i.e. hexagon, octagon, decagon, or other shape such as elliptical or oval. Concurrently, the inner wall 24 of the inner catheter 4 can also have a corresponding cross-sectional shape to match the surface profile of the outer wall 20 of sleeve 6, or it can be generally circular in shape as is depicted on FIG. 1a. In certain embodiments, it may be preferred to have a non-circular outer wall profile for the sleeve 6 in order to reduce the contact surfaces between the outer wall 20 of sleeve 6 with the inner wall 24 of the inner catheter 4 to thereby reduce the interfacial drag or frictional forces when these elements are slidably moved past one another.

FIG. 1a also shows a central cavity 26 of the sleeve 6 which the embodiment shown is at the axis of the sleeve 6. As is further shown in the figure, the central cavity 26 contains an element such as a guidewire 8 or other article or device useful in the treatment of a vessel occlusion which is also slidably positioned within the central cavity 26 of the sleeve 6. As is depicted in FIG. 1a, a guidewire gap exists between the exterior surface of the guidewire 8 and the inner wall 32 of the central cavity 6. While such a guidewire gap 30 need not necessarily be provided in all embodiments, nonetheless it is advantageously included in many embodiments thereby providing a guidewire gap 30 between the sliding surfaces of the guidewire 8 and the inner wall 32 of the central cavity and thereby facilitating the slidable movement of the guidewire 8 within the central cavity 26. When present such a guidewire gap 30 is between about 0.1 mm and 0.8 mm, and is preferably between about 0.2 mm and about 0.4 mm.

Yet again, as has been discussed generally above, although the embodiment for the guidewire 8 and the cross-sectional profile of the central cavity 26 is shown to be generally circular, it is to be understood that other non-circular configurations can be produced and are still considered to fall within the scope of the present invention. For example, the cross-sectional profile of the guidewire 8 can be any of a number of other possible configurations including for example, a strip which would produce a narrow rectangular cross-sectional configuration, a cross when the cross-sectional profile would be similar to two intersecting rectangles, a square, parallelogram, or polygonal shape such as a pentagon, hexagon, octagon, decagon, and the like. The use of a guidewire 8 having a profile other than the generally circular profile as depicted in FIG. 1a may provide several advantages. For example, a non-circular cross-sectional profile may be useful as providing multiple attachment points for a treatment article (not shown) which may be transported at the distal end of the guidewire and used to treat the occlusion or transverse the occlusion with a vessel in which the distal end 10 of the apparatus 1 is deployed, especially where such a vessel is a blood vessel. Alternately, the use of a solid guidewire having a non-circular exterior profile such as a square, or polygon may improve at the proximal end of the guidewire (not shown) the improved gripability of said proximal end of the guidewire either by human operator, or by a grip, handle, or other instrument which may be used to manipulate the guidewire 8. Further, such a non-circular exterior profile for the guidewire 8 may advantageously reduce the interfacial frictional contact between the exterior of the guidewire 8 and the interior wall 32 of the central cavity. Such reduction of friction may improve the operation of, and the handling of the guidewire 8 of the device 1.

It is to be expressly understood that while the embodiments depicted on the figures include both a sleeve 6 and guidewire 8, in certain preferred embodiment it is contemplated that one or both of these elements is absent.

It is also to be expressly understood that while the embodiments depicted on the figures include both a sleeve 6 and guidewire 8, in certain preferred embodiments it is contemplated that one or both of these elements may be replaced by a treatment article which may be useful in treating the vessel in which the distal end 10 of the device 1 is deployed, particularly an vessel occlusion such as a stent, a balloon tipped catheter, a dottering auger, a laser catheter as well as an atherectomy catheter and the like. Such may be readily deployed from within the interior of the inner catheter 4 or from the interior of the sleeve 6.

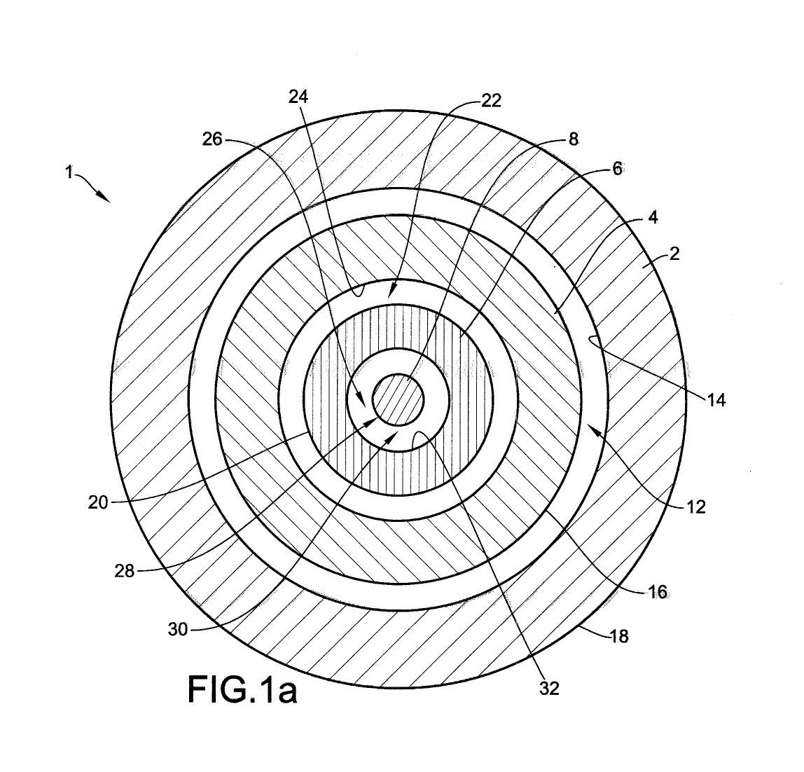

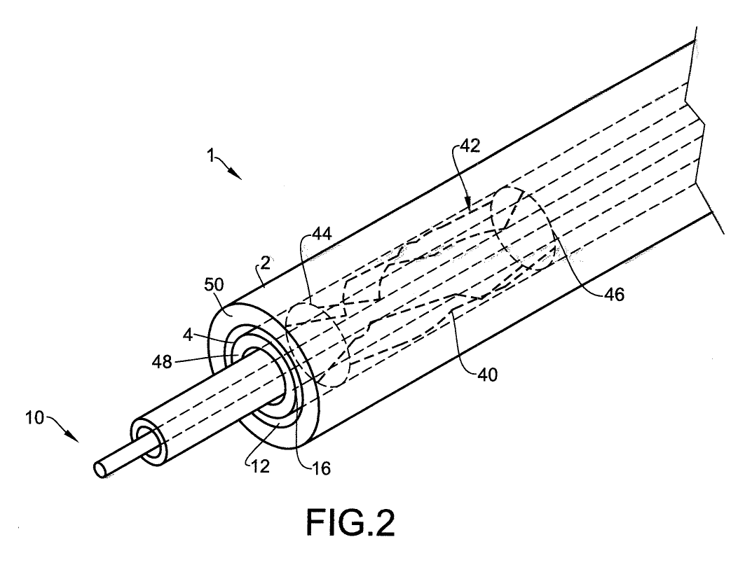

Turning now to FIG. 2, there is depicted in a perspective view the distal end 10 of the device 1 according to the present invention showing, in phantom, a partial view of an embodiment of the expandable cage 40 according to the present invention. As is depicted thereon, the expandable cage is seen to be within the interior of the outer catheter 2 and is associated with, preferably affixed to at least a portion of the outer wall 16 of the inner catheter 4. As is further visible in the figure, the cage 40 is shown in a collapsed, or compressed configuration and is seen to be positioned at least partially within the inter-catheter gap 12. As is also visible from FIG. 2, it is seen that the cage segment 42 has a front end 44, and a rear end 46. The front end 44 as is seen is spaced away, that is to say distally from the end 48 of the inner catheter and, in turn the rear end 46 of the cage segment is further distally located from the end 48 of the inner catheter. It is to be understood that the cage segment 42 need not necessarily be a specific element, such as a recess within the outer wall 16 of the inner catheter 4 although, conveniently such a circumferential recess is provided such that the elements forming the cage 40 can be collapsed at least partially to be retained within such a recess if present. However, as is shown in FIG. 2 no recess is necessarily present as such is often not required if the elements forming the cage 40 in their collapsed configuration are fittable within the inter-catheter gap 12 of the device 1.

The distance between the front end 44 of the cage segment and the end 48 of the inner catheter 4 can vary, but advantageously said front end 44 is coincident with the end 48, but more advantageously and preferably is positioned approximately 3 mm-10 mm, preferably 5 mm-7 mm from the end 48 of the inner catheter 4. Such an arrangement is preferred as it provides for part or parts of the cage 40 to be affixed, adjoined, adhered, or otherwise associated with a portion of the outer wall 16 of the inner catheter 4 at a position inwardly from the end 48 of the inner catheter 4 whereby when the end 48 of the inner catheter and the end 50 of the outer catheter are coincident, or coplanar as is depicted on FIG. 2, then it is assured that the cage 40 is collapsed and retained within the interior of the outer catheter 2 when the cage 40 is in a collapsed configuration. It is also to be understood that in such a preferred configuration, and being mindful of the fact that one or more elements of the cage 40 may be formed of flexible fibers or wires that when the end 48 and end 50 of the respective catheters are coincident that no part of the cage 40 or element thereof extends beyond either end 48 or end 50 of these respective catheters. Such provides a degree of safety in both the insertion of, and removal of the device 1 into a vessel and minimizes the risk of cutting or rupturing any of the vessel walls. In this manner, the distal end of the outer catheter 2 provides a sheath or a shield to the collapsed cage 40 and its elements.

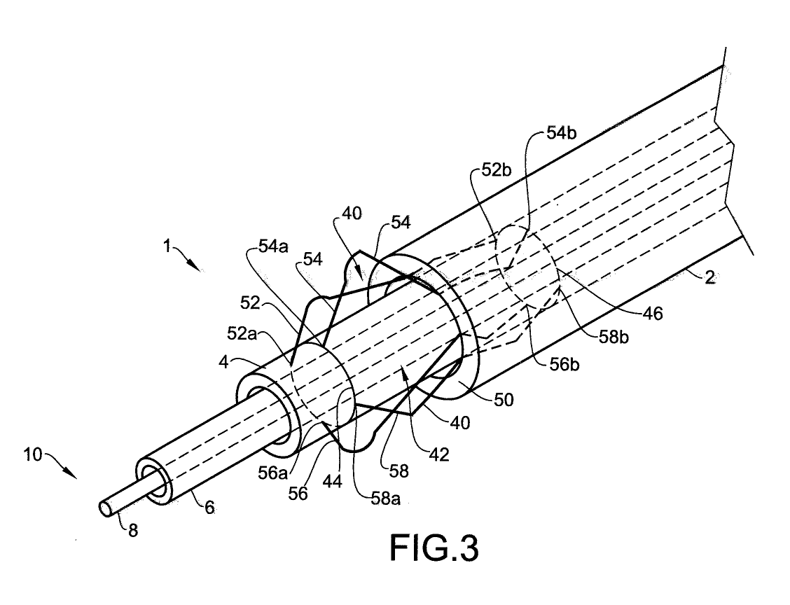

Turning now to FIG. 3, there is depicted the device 1 as depicted on prior FIG. 2 albeit in an alternative configuration. In the configuration depicted on FIG. 3, it is seen that the inner catheter 4 is partially extended outwardly, that is to say laterally and distally with respect to the outer catheter 2 thereby, exposing a portion of the cage segment 42 and corresponding portions of elements 40 of the cage outwardly beyond the end 50 of the outer catheter. As is visible in this configuration, wherein the cage segment 42 is only partially extended, that is to say the front end 44 of the cage segment extends beyond the end 50 of the outer catheter, but the rear end 46 of the cage segment 42 is retained within the interior of the outer catheter 2 and is still positioned proximally from the end 50 thereof, elements of the cage 40, namely cage legs 52, 54, 56, and 58 are seen to be in a partially “open” or expanded configuration. As is readily understood with reference to FIG. 3, the lateral movement of the inner catheter 4 exposes elements of the cage whereby portions thereof (52, 54, 56, 58) are allowed to flex, or to expand in a direction which is typically generally radially outwardly from the surface of the inner catheter 4. As is seen, each of the legs 52, 54, 56, 58 have a front end, namely 52a, 54a, 56a, and 58a which is affixed to the outer wall 16 of the inner catheter, or alternately may be anchored within portions of the inner catheter 4 through its outer wall 16, and similarly, each of the legs 52, 54, 56, and 58 have a proximal end corresponding to 52b, 54b, 56b and 58b which can similarly be attached to the outer wall 16 of the inner catheter, or anchored through the outer wall 16 of the inner catheter and into the inner catheter 4. The locus of the attachment points of the distal end of cage legs 52, 54, 56, 58 may be in a generally circular pattern such as is depicted on FIG. 3, but this is not necessarily required and configurations other than the circular arrangement as depicted in the figure are also possible. As is also to be understood, the opposite ends of the legs 52, 54, 56, 58 may also be affixed at their proximal ends at the rear end of the cage segment 46 either in a generally circular pattern as is disclosed on FIG. 3, but again this is not required and other configurations may exist. For example, the legs 52, 54, 56 and 58 may be positioned in an alternating pattern, that is to say wherein a first set of legs, e.g. 52, 56 are placed relative to the inner catheter 4 in a manner distinguishable from the second alternating set of legs, e.g. 54, 58. For example, the first set of legs 52, 56 may be positioned that their distal ends are positioned in a common plane however which is closer to or further from the distal ends of the second set of legs, 54, 58 which are positioned in a second common plane at a distance different from the end 50 than the plane of the first set of legs. Similarly, similar arrangement can also be produced for the proximal ends of alternating sets of legs. Additionally, although it is shown in FIG. 3 that the cage 40 comprises four legs which are approximately equally radially spaced apart from one another, that is to say are at approximately 90° intervals from the next adjoining leg, it is to be understood that greater, or lesser amounts of legs can form or be comprised as part of the cage 40. For example, at least two legs are typically, and preferably present in such configuration according to FIG. 3, but a multiplicity of legs, that is to say any number which is found to be useful according to the invention can be produced. In such configuration where a large multiplicity of legs are present, such is where for example 14 or more are present and such are optionally, but preferably equally radially spaced apart than a basket-type configuration might be produced. Most preferably however, a relatively smaller number of legs are required to form a preferred cage 40, that is to say between 3-8 or more preferably between 3-5, and most preferably about 4 legs are present and necessary to form preferred embodiments of the cage as are predicted in FIG. 3.

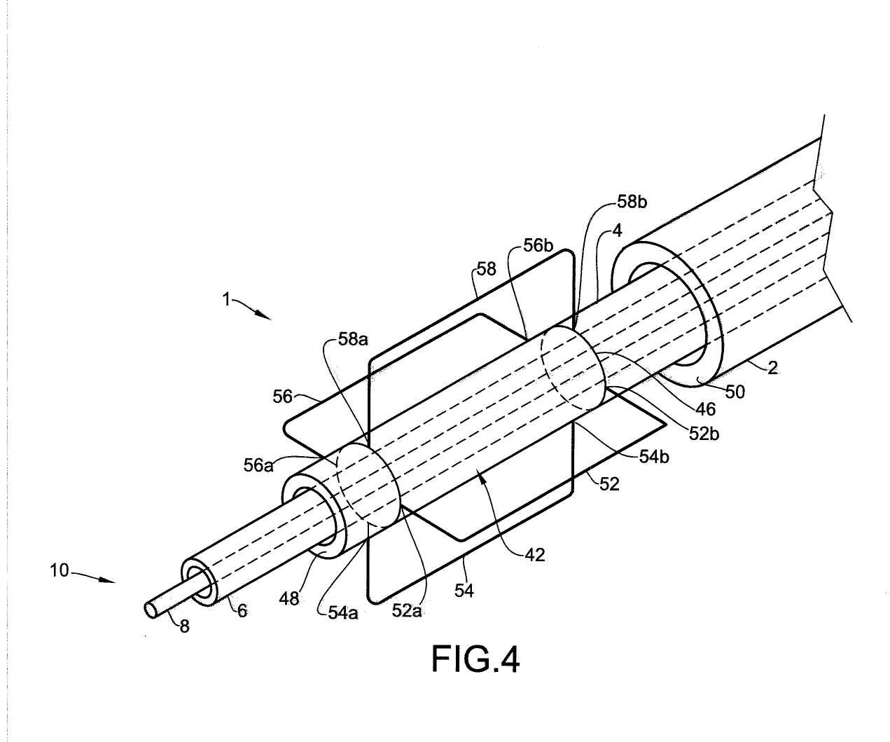

FIG. 4 depicts a further embodiment of the device according to the invention generally in accordance with FIGS. 2 and 3, albeit in the present embodiment the cage 40 is fully deployed. As is shown in FIG. 4, it is readily understood that the inner catheter 4 has been sufficiently transposed, or slid outwardly and distally from the interior of the outer catheter 2 such that the cage 40 and the cage segment 42 is now fully exposed beyond the end 50 of the outer catheter. In such configuration, the cage 40 and all elements thereof are free to expand to their maximum allowable configuration. This is represented in the figure by the fully extended legs 52, 54, 56, 58. As is more clearly seen from this figure, again these legs are anchored at both their respective distal ends 52a, 54a, 56a and 58 and at their proximal ends 52b, 54b, 56b, 58b such that each of the legs 52, 54, 56, 58 extend radially outwardly from the outer wall 16 of the inner catheter 4 in the region of the cage segment 42. Again, the cage segment is that portion of the outer wall 16 of the inner catheter 4 from which the elements of the cage 40 are permitted to expand. While not shown in the figure, it is to be understood that the configuration of the device 1 as depicted on FIG. 4, that is to say in its fully expanded configuration, can be easily returned to its partially expanded configuration as depicted on FIG. 3, and to its fully collapsed configuration as depicted on FIG. 2 by merely drawing or moving the inner catheter 4 in the proximal direction such that the inner catheter 4 and the cage 40 and its elements depending from the inner catheter 4 are retracted into the outer catheter 2 preferably where the inner catheter 4 is sufficiently retracted within the outer catheter 2 such that all parts or elements of the cage 40 are contained within the outer catheter 2. Alternately the outer catheter 2 may be moved with respect to the inner catheter 4 such that the inner catheter 4 and the cage 40 and its elements depending from the inner catheter 4 are retracted into the outer catheter 2 preferably where the inner catheter 4 is sufficiently retracted within the outer catheter 2 such that all parts or elements of the cage 40 are contained within the outer catheter 2. From the foregoing it is to be understood that only relative movement of the outer catheter 2 with respect to the inner catheter 4 is required, and one or both may be moved in order to result in the retraction of the cage 40 within the interior of the outer catheter 2.

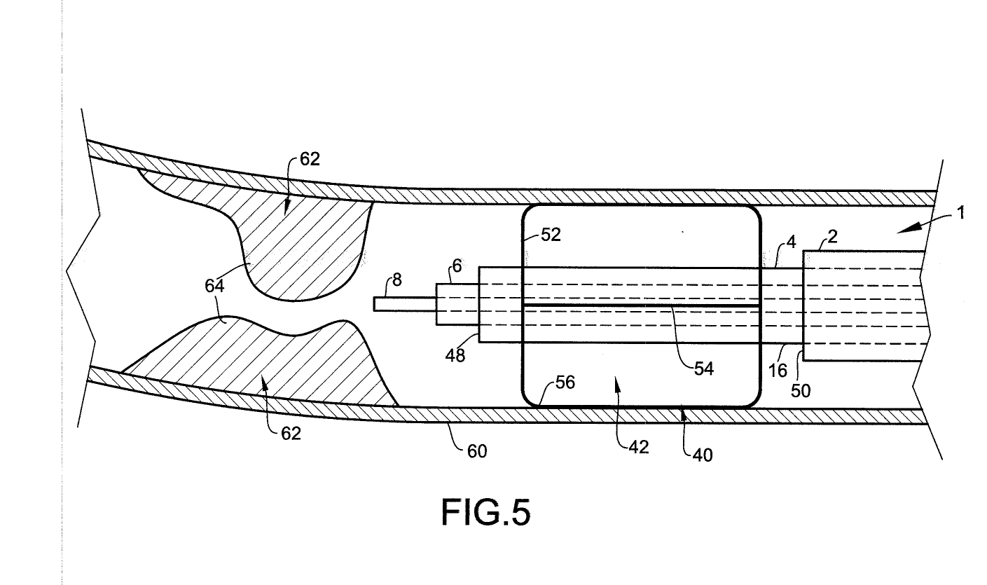

The placement within a vessel, and the operation of the device 1 according to the invention is described more fully with reference to FIG. 5.

FIG. 5 shows a cross-sectional view, depiction of the distal end of the device 1 according to the present invention as is generally depicted on FIG. 4 inserted within a blood vessel 60 of a human body or other patient wherein the device 1 is adjacent to the locus of an occlusion 62. As can be seen thereon, the elements of the cage 40, in this particular embodiment legs 52, 54, and 56, are fully extended outwardly and radially from the outer wall 16 of the inner catheter 4. In this configuration, the cage segment 42 is fully extended distally beyond the end 50 of the outer catheter. Due to the uniform configuration of each of the legs 52, 54, 56 (and not shown, 58) the expanded said legs act to automatically center the inner catheter 4 within the center line of the blood vessel 60 within which it is positioned. Thereby, any tool, article, treatment element or device, in this case the sleeve 6 and the guidewire 8 can be relatively precisely positioned at the approximate central axis of the section of the blood vessel 60 which is adjacent to the end 48 of the inner catheter 4. Such allows for the specific placement of the sleeve 6 and the guidewire 8 contained therein and now provides for a multiplicity of potential benefits. For example, due to the correct alignment and central placement of the inner catheter 4 by virtue of the expanded cage 40, a tool or device, such as guidewire 8 and a sleeve 6 can be placed and where desired, the guidewire 8 can be further distally extended outwardly from the end 48 of the inner catheter, and/or the sleeve 6 can be further distally extended beyond the end 48 of the inner catheter to attempt to pierce the deposits i.e. plaque deposits within the vessel at the locus of the occlusion 62 and/or potentially expand the vessel 60 at this locus and thereby cross the occlusion. Such may be particularly advantageous as, once the occlusion is crossed by the guidewire 8 and/or the sleeve 6, the inner catheter 4 may be withdrawn from the depicted position and be reintroduced into the interior of the outer catheter 2 thereby to collapse the cage and return it to a configuration as is depicted on FIG. 2. Thereafter, is contemplated that the inner catheter 4 and the outer catheter 2 can then be withdrawn from the human subject however, simultaneously allowing for the guidewire 8 and/or the sleeve 6 to be retained in position. The safe positioning of the guidewire, with the proximal end extending beyond the occlusion thereby very helpfully facilitates the reintroduction into the blood vessel a further device or apparatus which may be used to treat the human subject, or more specifically to treat the occlusion within the blood vessel. For example, angioplasty may be performed on the human patient, whereby the introduction of a conventional catheter having a balloon tip can be inserted very precisely and extended to the locus of the occlusion and the balloon expanded thereby distending the blood vessel 60 and thereby improving the blood flow through the vessel. Alternately an atherectomy procedure may be performed whereby the plaque deposits 64 may be scraped from within the blood vessel 60 and thereby also improving the circulation of fluids in particular blood through the vessel 60. Other treatment procedures which may be performed include but are not limited to laser angioplasty.

As has been discussed above, device 1 of the invention can also be used to precisely deliver a treatment article to the locus of a vessel occlusion after the distal end 10 of the device 1 is deployed, whereby the guidewire 8 and/or sleeve 6 are withdrawn after the distal end 10 of the device 1 is deployed and a treatment articles such as a stent, balloon tipped catheter, a dottering auger, a laser catheter or an atherectomy catheter is slidably inserted into the inner catheter 4 from the proximal end of the device 1 wherein such treatment article is advanced to the distal end 10 of the device from which it may be deployed in the vessel at the locus of the occlusion. Alternately, even prior to the placement of the device 1 within a human subject requiring treatment, the sleeve 6 and/or guidewire 8 may be substituted by such a treatment article which can be directly deployed from the inner catheter 4 after placement of the distal end 10 of the device at the locus of a vessel occlusion following the deployment of the cage 40 within the vessel.

The cage 40 is advantageously produced from a material which has a “memory”, that is to say a material which permits for the cage to reversibly assume the configuration of both a first collapsed shape, and a different expanded shape wherein the later shape is a larger expanded shape than the collapsed shape. Intermediate shapes are also contemplated. The collapsed shape corresponds to the configuration of the cage when the cage is fully enclosed within the outer catheter 2, and the expanded shape corresponds to the shape of the cage 40 when the cage is either allowed to expand outside of the body of a human subject, or alternately, the shape of the cage when the cage 40 is no longer enclosed by the outer catheter 2 but is enclosed within the confines of a vessel of a human subject. According to preferred embodiments, the geometry of the cage is such that the expansion of the cage 40 within the confines of a vessel of a human subject approximately equally distributes tensile forces from the outer wall 16 of the inner catheter 4 of elements of the cage 40 in its expanded shape within the vessel so to outwardly such that the inner catheter 4 is positioned nearly concentrically or concentrically within the vessel particularly blood vessel, viz. artery or vein. Such also provides a stabilizing and centering benefit to the catheter in the blood vessel, while the dimensions of the outer catheter 2 and the open nature of the cage 40 are such that placing of the device 1 does not block blood flow in the blood vessel 60. Such provides a “self-centering” benefit which allows for the accurate placement or extension of any treatment article or guide wire to extend from the interior of the inner catheter 4 to contact or transverse a vessel occlusion. Often, such occlusions are minimal at the approximate centerline (or axis) of the vessel at the locus of the occlusion which thus provides the least resistance to the insertion of a treatment article or guide wire into or across the occlusion. This minimizes the risk of undesired vessel wall perforation and potential harm to the patient. Additionally, unlike balloon catheter or other prior art devices which may be deployed in a vessel but whose use also necessarily totally blocks the flow of a fluid, i.e., blood during their deployment, the devices 1 of the invention including cages 40 do not completely stop blood flow even while they are placed as fluid in the vessel may continue to flow around the exterior of the outer catheter 2 and through the elements making up the cage 40.

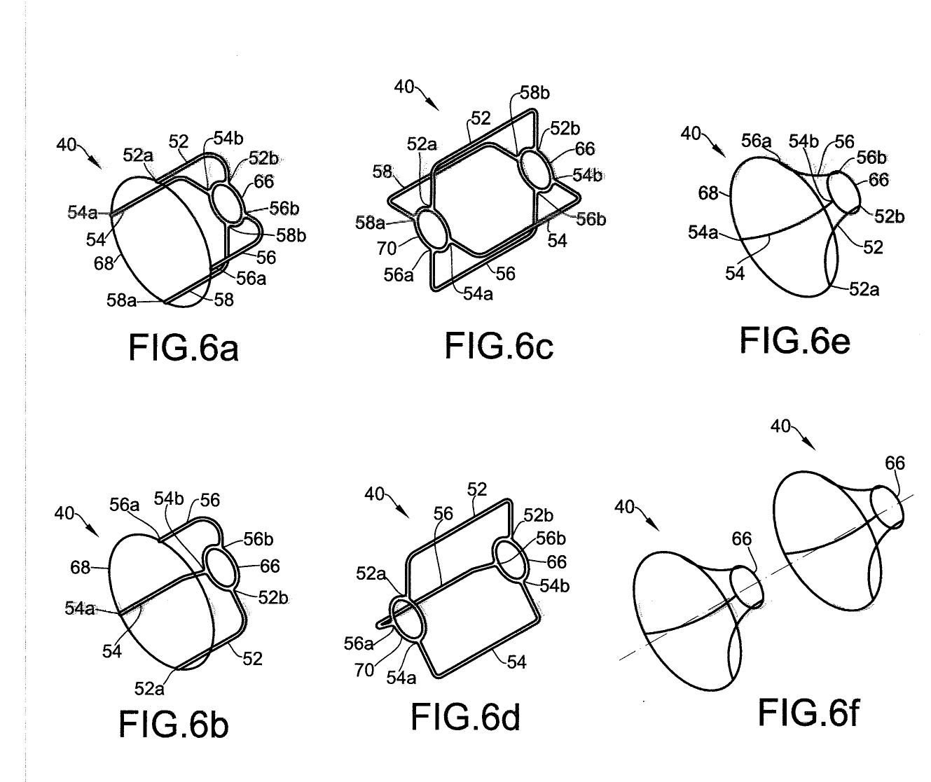

Various configurations for the cage 40 are foreseen and are considered to fall within the scope of the present invention. Turning now to FIG. 6a, there is shown an alternative embodiment of a cage 40 according to the present invention. As is seen thereon, there is provided a mounting collar 66 having radially extending therefrom in a common plane the proximal ends of four extendable elements, leg 52, leg 54, leg 56 and leg 58. As is seen in FIG. 6a, the mounting collar 66 is essentially circular in configuration and although not shown, it is intended that the dam over the mounting collar is appropriately sized so that it can be mounted about, or upon the peripheral outer wall 16 of the inner catheter 4 as depicted elsewhere in the figures. As is further seen, the corresponding proximal ends 52b, 54b, 56b and 58b of corresponding legs 52, 54, 56 and 58 extend radially outward initially in a plane coincident with that of the collar 66 and are spaced apart at approximately regular 90° intervals, with reference to the radius of the circular collar 66. Thereafter, it is seen that each of the legs bend outwardly from said reference plane in a direction distally from the collar 66 and the corresponding distal ends 52a, 54a, 56a, 58a of legs 52, 54, 56, and 58 terminate and are joined to a circular hoop 68 which has a diameter greater than that of the collar 66. The hoop 68 is affixed to the distal ends 52a, 54a, 56a, and 58a by appropriate fastening means, such that the hoop 68 is thus rigidly connected to the legs 52, 54, 56 and 58 at points which are approximately equidistantly spaced from adjacent points, that is to say at approximately 90° with reference to the radius of the hoop 68. While it is seen from FIG. 6a in the embodiment thereon that there are provided four legs 52, 54, 56, and 58 and that these are essentially equally spaced apart from one another, such is not a critical requirement of the present invention although it does depict one of alternate preferred embodiments. It is to be understood that variations on the configuration of the cage 40 are contemplated to fall within the scope of the invention including increased, or decreased numbers of legs; variations in the relative sizes of the corresponding elements of the cage 40, namely the legs, the collar 66 and the hoop 68 with respect to one another, and the like.

Turning the FIG. 6b, there is depicted an embodiment which is an alternate embodiment, yet retains many of the similar construction features and configurations of the preferred embodiment according to FIG. 6a. As is depicted on FIG. 6b, an alternative embodiment of a cage 40 is provided which shares many of the common elements of the cage embodiment according the FIG. 6a. As is shown thereon, there is depicted a collar 66 of a generally circular configuration having extended outwardly therefrom in a common plane the proximal ends 52b, 54b and 56b of legs 52, 54, 56, said distal ends 52a, 54a, 56a join to the outer circumference of a hoop 68. As visible from FIG. 6b, the proximal ends 52b, 54b, 56b initially extend outwardly from the collar 66 in a plane coincident with the collar 66 but thereafter bend outwardly in a common direction away from the plane such that they are essentially perpendicular to said plane and to said collar at their corresponding distal ends 52a, 54a and 56a where they are joined to the hoop 68. The plane defined by the collar 66 is essentially parallel to the plane defined by the hoop 68. Such a similar configuration is also depicted in FIG. 6a. Similarly to the depiction in FIG. 6a, in FIG. 6b the diameter of the collar 66 and that of the hoop 68 are different, with the diameter of the hoop being at least twice, preferably at least three times as large as that of the diameter of the collar. As is further seen from FIG. 6b, the placement of the legs 52, 54, and 56 are such that they are approximately equidistantly spaced apart with respect to one another where they join the collar 66 as well as where they join the hoop 68. Such has been similarly shown with respect to FIG. 6a, but in the embodiment according to the present figure there is approximately at 120° angle between the adjacent legs 52, 54, and 56.

With regard to FIGS. 6a, and 6b, while these figures do not depict the respective cages 40 as mounted upon an inner catheter 4 it is to be understood that the inner catheter 4 extends through the collar 66 which encircles a section of the inner catheter, and is used to affix the cage 40 thereto. Also, it is to be understood that the cage segment 42 as discussed with reference to other figures is in part defined by the dimensions of the cage, and more specifically that the collar 66 corresponds to the front end 44 of the cage segment, and that the point of intersection of the hoop 68 and the inner catheter 4 defines the rear end 46 of the cage segment, with the distance between the front end 44 and the rear end 46 of the cage segment corresponding to the cage segment 42 discussed in other figures. It is to be understood that cage segment need not be defined by actual connection points or contacts between the various elements forming a cage 40 but rather, that only one part or end of the cage 40 need be affixed to the inner catheter 4, and that the other end of the cage may be non-affixed and freely extend outwardly from the inner catheter and yet, the intersection of said other end and the transverse portion of the inner catheter can usefully define the other margin of the cage segment.

Turning now to FIG. 6c there is depicted a still further alternate preferred embodiment of the cage 40 according to the present invention. As is visible thereon, the cage 40 comprises a pair of collars 66, 70 one of said collars corresponding to the front end 44 of the cage segment, and the other of said collars corresponding to the rear end 46 of the cage segment. The collars are generally circular in configuration and both collars 66, 70 are of approximately equal size. While not depicted with reference to FIG. 6c, it is nonetheless to be understood that the diameter of the collars 66, 70 correspond approximately to the exterior diameter of the inner catheter 4 upon which the cage 40 is intended to be affixed. Extending in a plane coincident with each of the collars 66, 70 are four legs 52, 54, 56, 58. As is shown, the proximal ends 52b, 54b, 56b, 58b of corresponding legs 52, 54, 56, and 58 initially extend outwardly and depend from the collar 66 in a common plane as that of the collar 66, but bend outwardly in a direction ultimately perpendicular from the said plane defined by the collar 66 in a direction distally from the collar 66. The legs 52, 54, 56 and 58 continue to extend, then are seen to bend inwardly in a direction which is ultimately coplanar and coincident with the plane of the collar 70, to which the distal ends 52a, 54a, 56a and 58a of legs 52, 54, 56 and 58 are affixed. As is further seen in FIG. 6c, the legs 52, 54, 56 and 58 are approximately equally spaced apart, that is to say that they are relatively uniformly spaced at 90° angles with respect to one another, and further with respect to the plane defined by the collar 66 and/or the collar 70. Although the embodiment according to FIG. 6c depicts a preferred embodiment, it is to be understood that various variations on the cage 40 are possible and are contemplated to be within the scope of the invention. For example, the configuration and/or the number of legs extending from one or both of the collars 68, 70 may be altered and yet fall within the scope of the present invention.

FIG. 6d depicts an alternative albeit preferred embodiment of a cage 40 according to the present invention which is a variant of that cage 40 depicted on FIG. 6c. As is shown thereon, and similar to that in FIG. 6c, there are provided a pair of spaced-apart, equally sized collars 66, 70 which are connected to each other by three legs 52, 54, and 56 wherein the proximal ends 52b, 54b, 56b of each leg is affixed to the outer circumference of the collar 66, which also defines the front end 44 of the cage segment, and the distal ends 52a, 54a, 56a are of each of the legs 52, 54, 56 are affixed to the other collar 70, which also defines the rear end 46 of the cage segment. Again, similarly to the embodiment depicted in FIG. 6c, the portions of the legs 52, 54, 56 which are adjacent to their proximal ends 52b, 54b, 56b which depend from the collar 66 initially extend outwardly from said collar 66 in a plane coincident with said collar 66 but after a short distance, are seen to bend in a direction away from and essentially perpendicular to the plane defined by the collar 66. These legs 52, 54, and 56 continue to extend in a distal direction until they again bend, inwardly and in a direction essentially parallel to the plane defined by the collar 66 until their distal ends 52a, 54a and 56a terminate where they depend from the other, second collar 70 where as is seen their distal ends 52a, 54a, 56a are also depicted as extending radially outwardly from the second collar 70. As is visible from FIG. 6d it is also apparent that the legs, as affixed to both the collar 66 and the second collar 70 are approximately equally spaced apart with respect to each other, namely in that the legs are spaced apart approximately 120° with respect to one another, with reference to the radius of the collar 66 and/or collar 70.

Turning now to FIG. 6e there is depicted a still further embodiment of a cage 40 according to the present invention, which is also amongst preferred embodiments. As is depicted thereon, the cage 40 comprises a collar 66 which is generally circular in configuration and has extending in a direction laterally therefrom three spaced-apart legs 52, 54, 56. The proximal ends 52b, 54b and 56b depend from the collar 66 and initially extend in a direction which is essentially perpendicular to the plane defined by the collar 66 but, are seen to bow, or curve outwardly in a distal direction until the legs 52, 54, 56 terminate at their corresponding distal ends 52a, 54a, 56a, said distal ends depending from a hoop 68. While not depicted in FIG. 6e, it is to be understood that the dimensions of the collar 66 are such that it is adept to be mounted upon the exterior of the inner catheter 4, and thus, the inner diameter of the collar 66 is approximately equal to or perhaps slightly larger than the diameter of the inner catheter. The diameter of hoop 68 is preferably at least as large as that of the collar 66, more preferably is at least 1.5 time, more preferably 2 time, still more preferably 2.5 times, and most preferably at least 3 times the diameter of the collar 66. Also preferably, as is shown in FIG. 6e, the plane defined by the hoop 68 is parallel to the plane defined by the collar 66. Additionally, as depicted in FIG. 6e, the legs 52, 54, and 56 are all of equal length although it is contemplated that one or more legs can have different lengths and/or dimensions. As is seen from the embodiment depicted on FIG. 6e, in comparison to that of FIG. 6b, several common elements are present, in both, a collar 66 is provided and a hoop 68 is provided and wherein the plane of the collar 66 parallel to the plane defined by the hoop 68. Further, in each of these embodiments three legs are provided although, the primary distinction resides in that the direction of curvature of the legs 52, 54, and 56 extending between the collar 66 and the hoop 68 differ between these two embodiments.

Turning now to FIG. 6f there is depicted a further embodiment of the present invention which, for illustrative purposes utilizes two cages 40 according to the present invention, said cages being in accordance with the embodiment depicted on FIG. 6e. Whereas it has been to this point discussed that the device of the present invention comprises a cage 40 it is to be understood that the device may also include a plurality of cages such as are depicted in FIG. 6f. Each of the cages 40 depicted on FIG. 6f are essentially identical to that described with reference to FIG. 6e. Similarly, the collars 66 of each cage are essentially circular, and are dimensioned such that they are adapted to be mounted upon the inner catheter 4, preferably around a portion of the outer wall 16 of the inner catheter 4. In accordance with the embodiment of FIG. 6f, it is seen that two (or more) cages can be applied to a single inner catheter 4 and mounted thereupon wherein a first cage and a second cage are both mounted upon an inner catheter in a spaced-apart type relationship as shown in FIG. 6f. That is to say, that one of the two cages is mounted upon the inner catheter 4 in a position which is proximal and the second (as well as further) cages are provided in a direction closer to the distal end than said first cage. While not shown in FIG. 6f, it is also to be understood that various alternate arrangements, and use of cages having different configurations are also contemplated as being a part of the present invention. That is to say, two or more cages having different geometries and/or configurations are also considered to be useful with respect to the multiple cage configuration suggested by FIG. 6f. For example, any combination of the cages depicted on FIG. 6a through 6e can be combined in any order with respect to one another upon a single inner catheter 4.

Returning briefly to FIG. 4, the applicant points out that the embodiment shown at that figure shows a still further alternative embodiment of a cage 40 according to the present invention which can be contrasted to similarly configured cage 40 according to FIG. 6c. The distinction between the embodiment according the FIG. 4 and the cage 40 depicted thereon is that the legs 52, 54, 56 and 58 of that cage are embedded into the side wall of the inner catheter 4 and do not comprise a collar 66 and/or collar 70. Rather, the proximal ends 52b, 54b, 56b and 58b, as well as the distal ends 52a, 54a, 56a and 58a of corresponding legs 52, 54, 56 and 58 are embedded or anchored directly into a portion of the outer wall 16 of the inner catheter 4. Thus it is clearly contemplated that collar-less configurations of cages 40 are contemplated as falling within the scope of the present invention. Further, the positioning of the cage 40 as depicted on FIG. 4 also represents a preferred positioning of any of the alternative cages as described with reference to FIG. 6a-6f with regard to placement upon the inner catheter 4 with respect to the end 48 of said inner catheter 4. Of course, such a placement, albeit highly preferred, is to be understood not by way of limitation but merely as a preferred embodiment.

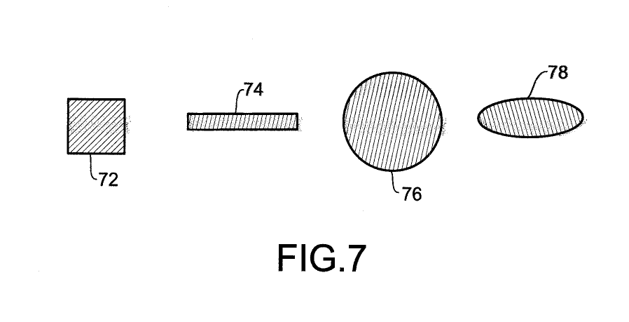

FIG. 7 depicts a plurality of cross-sectional configurations of the elements which may be used to form the cage which are amongst preferred embodiments of the elements which may be used to make up cage 40 as described herein. As can be seen thereon, various cross-sectional profiles are contemplated and the four profiles comprise preferred embodiments. 72 depicts a cross-sectional profile with essentially square cross-section. Cross-sectional profile 74 depicts a profile which is essentially rectangular in cross-section. Cross-sectional profile 76 depicts an essentially circular cross-section. Cross-sectional profile 78 depicts an essentially elliptical cross-sectional profile. Each of these cross-sectional profiles are preferred for use in the construction of part, or all of the elements used to form a cage 40 according to the present invention. Cross-sectional profile 72, 74 are advantageous in that they provide square corners or vertices which advantageously may be used to temporarily anchor the contact of points or parts of an expanded cage 40 to a vessel 60 wall without damaging, or tearing the vessel wall. Cross-sectional profile 76, 78 are curved, or smooth edged and provide no corners or vertices and also may be used to define parts or all of the elements used to form a cage 40 according to the invention. These two profiles have however smooth edges and provide no corners or vertices which may risk undesired puncture or tearing of the blood vessel walls. Each of the foregoing profiles has certain advantages which may be enjoyed, and preferred for use according to appropriate conditions wherein the device of the invention is intended to be used.

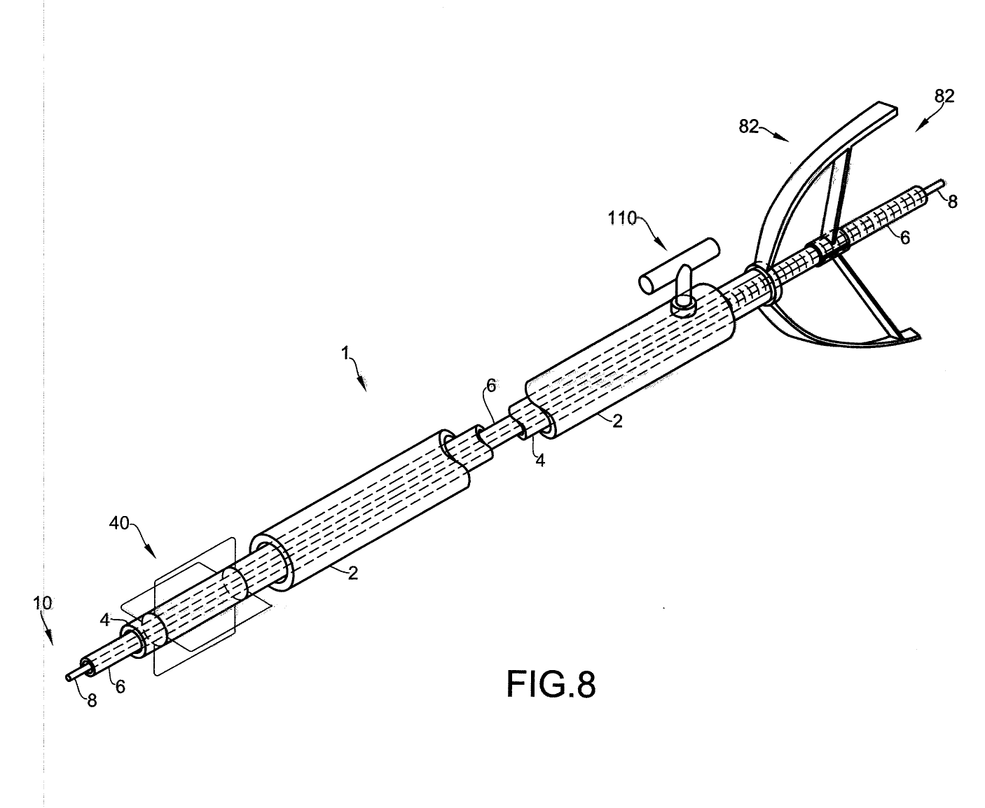

FIG. 8 depicts a further preferred embodiment of the device 1 according to the present invention. FIG. 8 depicts a preferred embodiment of the device which includes a representation of a complete device, that is to say the device having at the distal end thereof a cage 40 and at the proximal end thereof a means for controlling the operation of the device which is particularly adapted to manual control by a surgeon, or healthcare provider, or adapted for automatic or semiautomatic control by a machine or robot. It is to be noted that the elements depicted on FIG. 8 as well as the following figures are not necessarily to scale.

With reference now to FIG. 8, at the proximal end 10 of the device 1 is depicted the guidewire 8 extending outwardly therefrom the sleeve 6, concurrently extending outwardly therefrom the inner catheter 4, and still further extending outwardly therefrom the outer catheter 2. As depicted in FIG. 8, the view of the end thereof essentially corresponds to that already described with reference to FIG. 4. At the proximal end of the device 80 as is seen thereon the device 1 depicts mounted thereon a preferred embodiment of a control device 82 which may be used to control the operation of the device 1. Additionally, a control device 82 may be used to facilitate the insertion of, and withdrawal of portions of the device 1. In FIG. 8, the control device 82 is fixed onto outer catheter 4 via the mounting collar 84. Notwithstanding the specific embodiments presented herein on FIG. 8, it is nonetheless to be understood that a control device may be affixed to or associated with any other part of the device 1, e.g, the inner catheter 4, sleeve 6, guidewire 8 in order to provide a means for advancing such other part or parts of the device relative to other parts of the device 1.

Notwithstanding the foregoing, it is to be expressly understood that the device 1 may be operated without a control device 82 and that the cage 40 may be deployed manually.

In embodiments with a control device 82, (or absent such a control device,) the proximal end 10 may be inserted into a vessel, such as a blood vessel and the control device 82 may be used to advance the sleeve 6 in small increments towards an occlusion within the vessel after the cage 40 has been previously deployed. As the sleeve 6 approaches the occlusion it may encounter resistance to movement and has a tendency to push back in the opposite direction, viz., distally. The flexible disk 85 is desirably sufficiently rigid to prevent or at least retard this push back but is at the same time, sufficiently flexible to permit the operator of the device 1 to pull the sleeve 6 out of the catheter when it is not needed. When present, the control device 82 conveniently assists the operator to push the sleeve 6 into the locus of the occlusion, and after the sleeve 6 has been extended as far as desired or as far is possible, the guidewire 8 may be extended by the operator to push it through the occlusion within the vessel. All this time, the deployed cage 40 keeps the sleeve 6 and guidewire 8 centered within the vessel adjacent to or at the locus of the vessel occlusion.

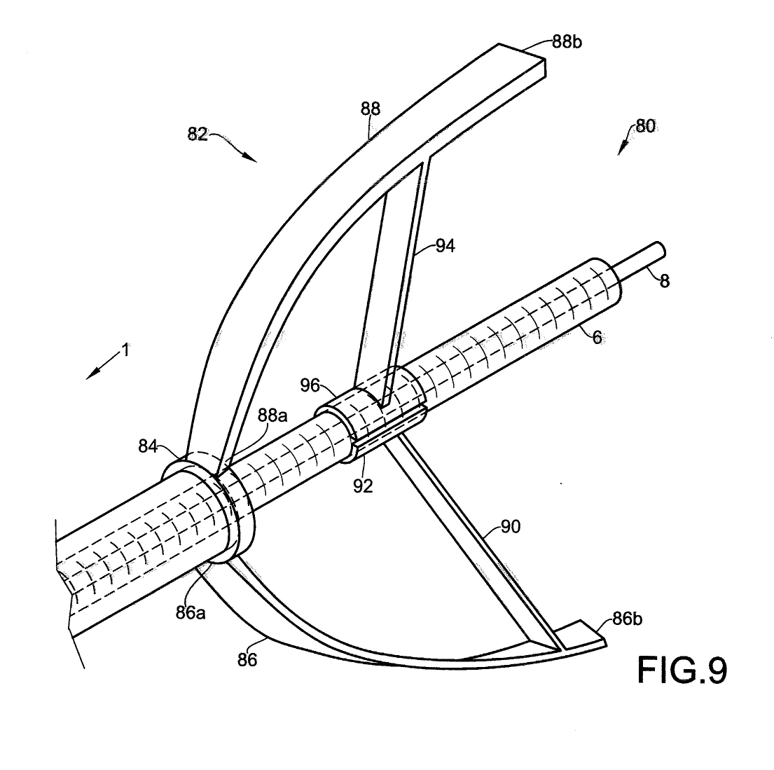

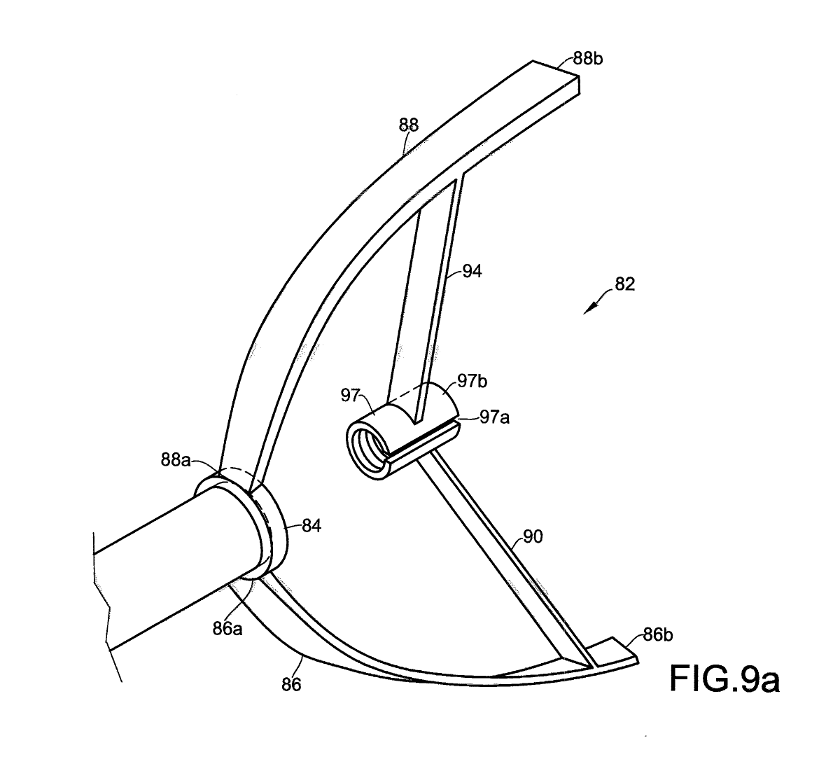

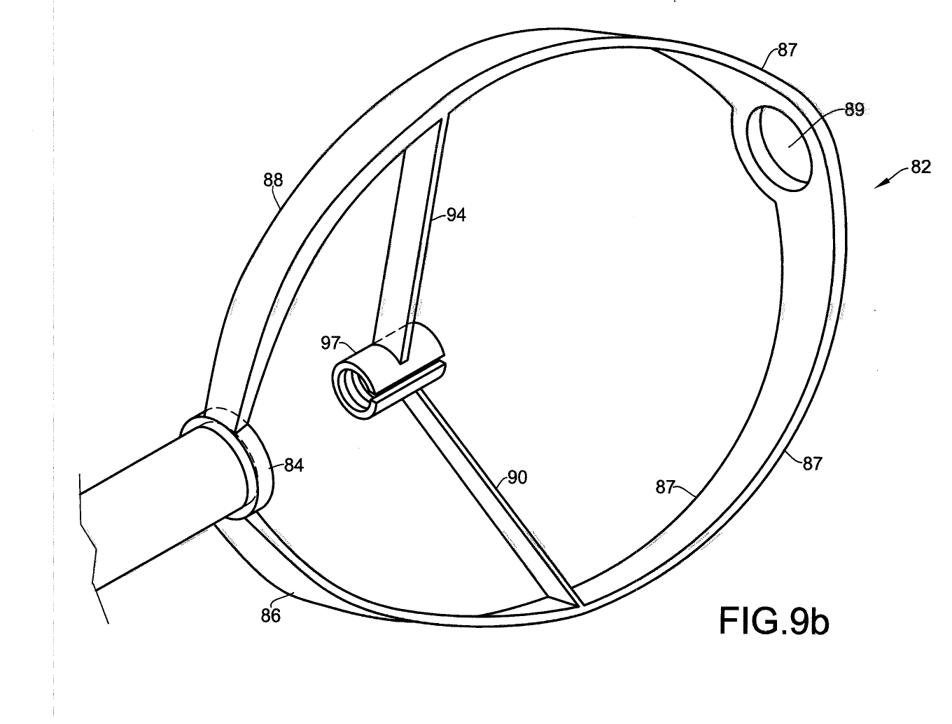

Turning now to FIG. 9, there is depicted in greater detail the features of the control device 82. As is shown thereon, the control device 82 comprises a seating collar 84 having extending outwardly and in the direction approximately therefrom a pair of control grips, a first control grip 86 having a connecting end 86a depending from the seating collar 84, and an opposite terminal end 86b at the opposite end thereof. A similar second control grip 88 is also provided which similarly has a connecting end 88a depending from the seating collar 84, said control grip 88 extending to and ending at a terminal end 88b. As depicted in FIG. 9, it is intended to be understood that the control grips 86, 88 are intended to be moveable with respect to both the seating collar 84 and with respect to the device 1 and more specifically are intended to be moveable both towards, and away from both the inner catheter 4 and the outer catheter 2. This is easily accomplished, for example by having a human operator, such as a surgeon manually grip the control grips 86, 88 in the palm of their hand and by light compression cause the control grips 86, 88 to be compressed in the direction wherein the respective terminal ends 86b, 88b, move closer or towards the outer catheter 2 and the inner catheter 4. However, by releasing said control grips 86, 88, said flexible control grips move, or spring outwardly and away from the outer catheter 2 and the inner catheter 4. The control device 82 also comprises an advancing means which is used to advance the sleeve 6 with respect to the inner catheter 4. More specifically, the advancing means provides for lateral motion, that is to say a sliding in-and-out motion, whereby the sleeve, can be moved within the inner catheter 4. In the depicted embodiment according to FIG. 9, the advancing means includes a pair of linkages, a first linkage 90 extending from a portion of the control grip 86 at a point proximate to the terminal end 86 therefrom and having another end depending from a first split ferrule 92 and a similar second linkage 94 having one end depending from the second control grip 88 at a point reasonably proximate to the terminal end 88b thereof, with the other end of the second linkage 94 depending from a second split ferrule 96. As can be seen from the figures, the first linkage 90 is seen to depend from the first control grip 86 at an angle relation thereto such that the opposite end of the first linkage 90 is directed distally. Similarly, the second linkage 94 depending from the second control grip 88 is also seen to be angled, such that its end depending from the second split ferrule 96 is also seen to be directed distally with respect to the second control grip 88. In this manner, it will be understood that compression of the control grips 86, 88, i.e. movement towards one another, will urge the ends of the first linkage 90 and the second linkage 94 which are correspondingly attached to first split ferrule 92, and second split ferrule 96 in a relative distal direction which action causes the first split ferrule 92 and second split ferrule 96 to approach one another and thereby grip a portion of the sleeve 6 and thereby urge it to move in slideable manner within the inner catheter 4 in distal direction. Conversely, release of the control grips 86, 88 allows their expansion, and retraction of the first and second linkages, correspondingly the retraction of first and second linkages 90, 94 and first and second split ferrules 92, 96 away from the sleeve 6.

FIG. 9a depicts a control device similar in most respects to prior FIG. 9 wherein the depiction of the sleeve 6 and the inner catheter 4 are omitted for the sake of clarity. A distinguishing feature which is visible on FIG. 9a, is that the first split ferrule 92 and the second split ferrule 96 described with reference to FIG. 9 are herein replaced by a single gapped ferrule 97 which as is visible on FIG. 9a is generally cylindrical and which has a substantially continuous sidewall 97b which is however intersected by a single gap 97a. The gapped ferrule 97 is also depends from first and second linkages 90, 94 which correspondingly depend from control grips 86, 88. As is visible from the figure, the first and second linkages 90, 94 depend from the gapped ferrule 97 such that the linkages 90, 94 are preferably diametrically opposed to each other across the sidewall 97b of the gapped ferrule 97 and further, that the gap 97a is approximately at a midpoint therebetween. In this arrangement, the maximum compression of the sidewall 97b can be achieved, and that the gap 97a minimized thereby decreasing the effective diameter or cross-sectional dimension of the gapped ferrule 97 while the control grips 86, 88 are compressed, and conversely, when the control grips 86, 88 are released, the compression of the gapped ferrule 97 is also released and the gapped ferrule 97 returns to an uncompressed state and to its maximum effective diameter or cross-sectional dimension. An advantage of using to any gapped ferrule 97 is that potential alignment issues between the portions of the split ferrules such as depicted in FIG. 9 are virtually eliminated. In operation, and with respect to FIG. 9a, it will be understood that compression of the control grips 86, 88, i.e. movement towards one another, will urge the ends of the first linkage 90 and the second linkage 94 which are correspondingly attached to opposite sides of the gapped ferrule 97 in a relative distal direction which action causes the gapped ferrule 97 to be compressed and thereby grip a portion of the sleeve 6 and thereby urge it to move in slideable manner within the inner catheter 4 in distal direction. Conversely, release of the control grips 86, 88 provides for the release of compression on the gapped ferrule 97, correspondingly the release of the sleeve 6 by the gapped ferrule.

FIG. 9b illustrates a further embodiment of a control device 82 according to the invention which is similar in many respects to the embodiment of the control device 82 as depicted on FIG. 9a. The control device 82 of FIG. 9b differs from the control device 82 of FIG. 9a in that the control grips 86, 88 are configured such that continue to extend that ultimately join one another in order to form a continuous grip bridge 87 which results in a continuous, generally elliptical grip handle as a shown on the figure. A control device having such a continuous grip handle which is meant to be understood as the combination of elements 86, 87, and 88 provides the advantage of an increased grippable surface which may improve the grippability or graspability of the control device 82 as well as providing additional flexible elements, namely continuous grip bridge 87 which may work with integrally interconnected or integrally formed control grips 86, 88 in order to provide improved outward flexure, that is to say outward flexural bias of the control grips 86, 88. Such may be provided to provide improved control of the movement of the sleeve 6 with respect to the inner catheter. Additionally, preferably, the continuous grip bridge 87 may be conveniently provided with a passage 89 at a portion of the continuous grip bridge 87 through which a portion of either the sleeve 6 and/or the guidewire 8 may project, or is expected to be projected. Such may be useful in facilitating the movement of, and/or the replacement of portions of the device 1 according to the present invention. Preferably, the dimensions of the passage 89 are such that said passage 89 is of sufficient size, keep your e.g., diameter or cross-section to provide for the unimpeded withdrawal of, or insertion of portions of the sleeve 6 and/or guidewire 8.

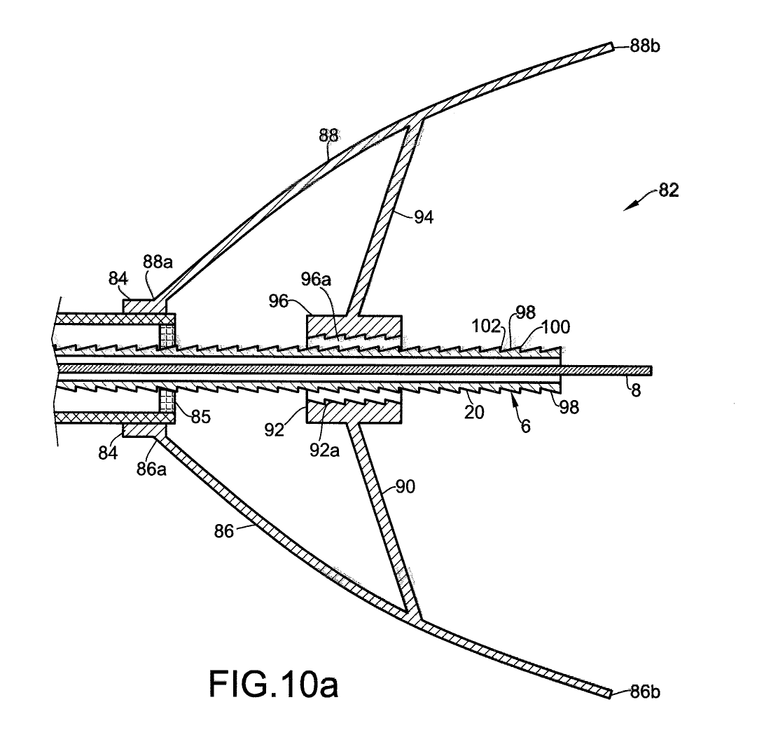

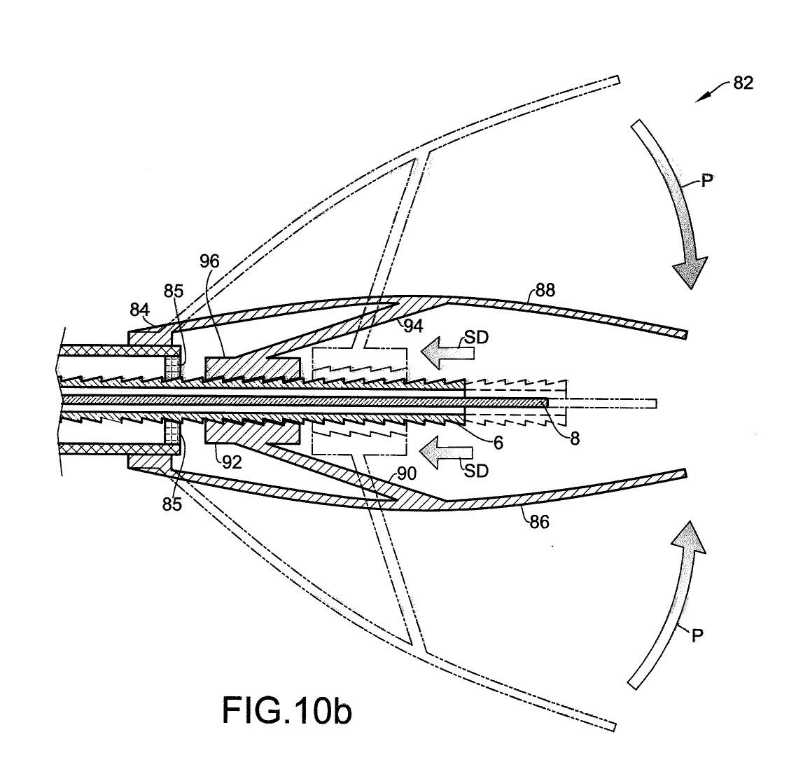

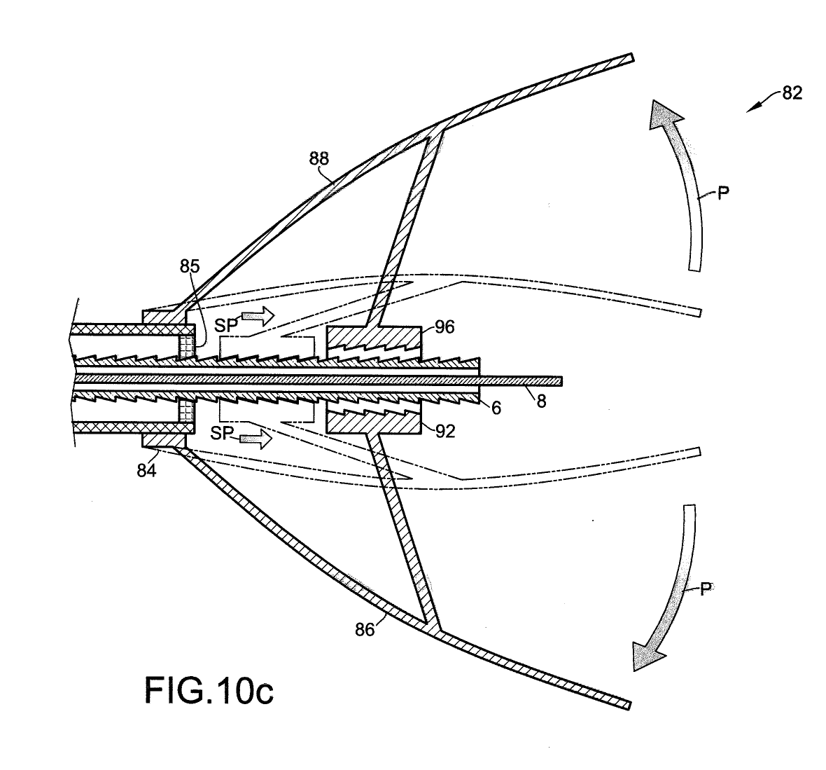

Turning now to FIGS. 10a, 10b and 10c, the operation of the control device 82 will be described in more detail. FIGS. 10a, 10b and 10c depict the control device according to FIG. 9 in three different modes or operating states

With reference to FIG. 10a, as is depicted thereon there is visible control grips 86, 88, their depending first linkage 90 and second linkage 94, and their corresponding depending split ferrule 92, and second depending split ferrule 96. Also shown in further detail is the sleeve 6 and in this cross-sectional view it is seen that this portion of the sleeve 6 comprises a series of evenly spaced frustoconical segments 98, each frustoconical segment 98 being repeated in a uniform pattern along the length of the portion of the sleeve 6. Each of the frustoconical segments 98 comprises a base portion 100 wherein the respective frustoconical section 98 is at its greatest diameter, which then tapers inwardly towards the guidewire 8 to its opposite terminal end or peak 102 which defines the minimum diameter of each respective frustoconical segment. As is clearly visible from the figure, such an arrangement is regularly repeating, with the direction of the tapered section being directed to the distal end of the device 1. With reference now to the split ferrules 92, 96, as is seen the inner walls of the respective split ferrules 92a, 96a are profiled to have a “reverse” frustoconical segment, here a plurality of “reverse” frustoconical segments are shown on their respective inner wall 92a of the first split ferrule, and the inner wall 96a of the second split ferrule 96. These profiles are intended and are adapted to be in a mating relationship with corresponding frustoconical segments 98 when the control grips 86, 88 are compressed which in turn causes the split ferrule 92, and 96 to advance and contact the outer wall 20 having been provided with frustoconical segments 98. Once engaged, and with any further pressure, the split ferrule 92, 96 urges the sleeve 6 to be moved in a distal direction wherein at the opposite end thereof, namely at the distal end 10 of the device 1 the sleeve 6 and the guidewire 1 are urged and/or moved in the direction beyond the end 48 of the inner catheter 4. In such a manner, the device according to the invention, and in particular utilizing the control device 82 can be used to carefully and accurately extend the sleeve 6 and with it where desired the guidewire 8. The relative position of the sleeve 6 with regard to the seating collar 84 is also additionally maintained by flexible disk 85 which spans the seating collar 84 and comes into interfacial contact with the frustoconical segments 98 of the sleeve 6. As can be understood with reference to the drawing, a grip-like contact is established by a compression fit between the flexible disk 85 and a portion of the sleeve 6 and this compression fit is maintained at all times. It is to be pointed out that this compression fit is not unduly strong but can be overcome by manually withdrawing the sleeve 6 by pulling it into a proximal direction, or by advancing the sleeve 6 in the manner described above using the control device 82.

FIG. 10B illustrates the state of the control device 82 wherein the control grips 88, 86 are shown in a compressed relationship, viz., the control grips 86, 88 are moved in the direction of arrows “P”. In such a state, as is seen thereon the split ferrules 92, 96 are shown engaging a portion of the sleeve 6 via mating with portions of frustoconical segments 98 on the periphery of the sleeve 6 which are simultaneously engaged by the first split ferrule 92 and second split ferrule 96. Such urges or moves the sleeve laterally 6 in a distal direction with reference to the device 10. Such a motion causes the sleeve 6 to slide within the inner catheter 4, in the direction indicated by arrows “SD”. The engagement of the split ferrules 92, 96 also deny the retraction of the sleeve 6 when the mating with portions of frustoconical segments 98 engage corresponding mating portions of the first split ferrule 92 and second split ferrule 96. Of note is that features of the drawing depicted in phantom corresponds to the arrangement depicted in FIG. 10a, which is provided for purposes of illustration and comparison. The control grip 86, provides a grippable or graspable element of the device 1 which can be used to insert or retract the inner catheter 4 within the human subject independently of the motion of the outer catheter 2 and/or sleeve 6.

FIG. 10C depicts the condition of the control device 82 wherein the control grips 88, 86 are shown in a non-compressed relationship, viz., the control grips 86, 88 are allowed to move in the direction of arrows “P”. In such a state, as is seen thereon the split ferrules 92, 96 are shown released from a portion of the sleeve 6 via mating with portions of frustoconical segments 98 on the periphery of the sleeve 6 which allows for lateral motion, including insertion or withdrawal of the sleeve 6 laterally in a distal, but usually in a proximal direction with reference to the device 10. Such a motion permits the sleeve 6 to slide within the inner catheter 4, in the direction indicated by arrows “SP”. The release of the previously engaged split ferrules 92, 96 permit for the retraction of the sleeve 6, and/or permits for the sleeve 6 to remain in the same relative position with respect to other parts of the device 1, especially with respect to the inner catheter 4. If desired or necessary, following such a release of the previously engaged split ferrules 92, 96, the user or operator of the device, viz., medical personnel, may compress the control grips 88, 86 again bringing the split ferrules 92, 96 into engagement and thus force the sleeve 6 to slide within the inner catheter 4. Of note is that features of the drawing depicted in phantom corresponds to the arrangement depicted in FIG. 10b, which is provided for purposes of illustration and comparison.

It is to be understood that the foregoing mode of operation described with reference to FIGS. 10A-10C is equally relevant to configurations of the control device 82 which includes a gapped ferrule 97 as disclosed on FIG. 9a, as well as a continuous grip handle, viz., the combination of elements 86, 87, and 88 as disclosed on FIG. 9b.

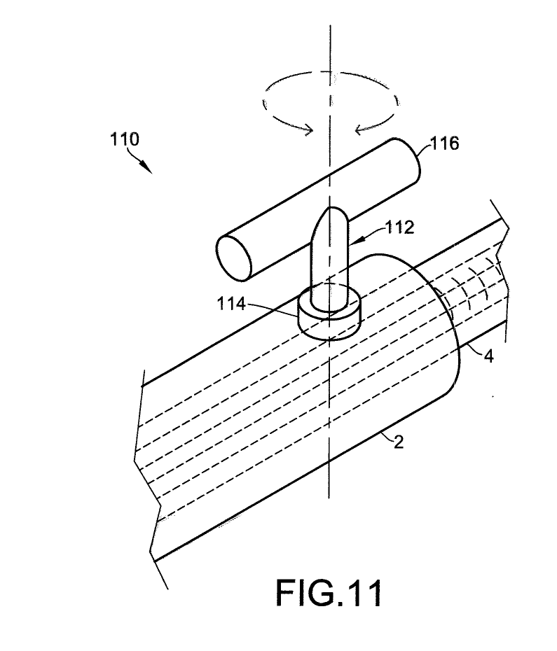

FIG. 11 depicts an optional, locking means 110 which may be provided to the device 1 of the invention. The locking means 110 includes a movable post 112 which passes through a collar 114, which locking collar is mounted upon a portion of the outer catheter 2. The locking collar 114 has a passage or borer passing therethrough into which the post 112 may be moved inwardly, and outwardly with respect thereto. Such may be provided for example by an interference-type fit, a pair of a mating threads on both the post 112 and an inner surface (not shown) on the collar 114, were any other means. It is only required to that relative motion between the post 112 and the collar 114 be provided. Although not shown in the figure, it is to be understood that the collar 114 is mounted over a corresponding passageway or bore (not shown) which extends through at least the outer catheter to. An end, also not shown of the post 112 is adapted to come into physical contact against the inner catheter 4 and one in a locking condition is used to maintain the relative positions of the outer catheter 2 with respect to the inner catheter 4, which allowing the sleeve 6 and guidewire 8 to be moveable with respect to one another and with respect to the inner 4 and outer 2 catheters. Such may be advantageous during certain parts of a procedure used to remove the occlusion in any vessel, such as a blood vessel, using the device 1 of the present invention, as a control means, e.g. a pair of control grips 86, 88 may be used to advance the sleeve into the vessel within the body of a patient. Of course, such may also be performed without any control means being present or used with the device 1, but may be performed wholly manually by manual manipulation of by an operator. To facilitate the positioning of the post 112 with respect to the collar 114 and the further elements of the device 1 of the invention, a handle 116, here in the shape of a cylindrical rod is used to provide a “T”-shaped configuration in conjunction with the post 112. A handle 116 and maybe easily gripped by one or more fingers at me and can be used to position at the post 112 with respect to the collar 114. In one configuration, the handle 116 and attached post 112 are both affixed with respect to one another, and these elements are rotatable within and with respect to the collar 114. The post 112 may be fully removable from the collar 114, or maybe nonremovable with respect to the collar 114.

The articles of the invention may be used to treat both human and animal subjects in need of treatment. The articles of the invention advantageously used to treat occluded blood vessels, but can be used to treat other bodily vessels in human and animal subjects which may form, or which are prone to form occlusions. The articles of the invention may be used therapeutically, that is to say to treat an existing occlusion within a vessel, or prophylactically, that is to say to treat a vessel where no occlusion exists but where an occlusion may form. The articles of the invention may also be used to place articles or devices, e.g., stents, filters or other devices within a vessel, e.g., blood vessel of a human and animal subject.

In a further aspect of the invention there is provided a process for the treatment of vessels, particularly occlusions in said vessels and especially particularly chronic total occlusions which process comprises the steps of:
positioning the distal end of a device which comprises an outer catheter and an inner catheter, and a treatment article, including but not limited to a guide wire, a balloon tipped catheter, a dottering auger, or other element, slidably deployable from the interior of said inner catheter, said inner catheter having disposed on a part of its outer surface a collapsible cage, wherein the said inner catheter and cage are adapted to be slidably deployed from the interior of said outer catheter within a vessel adjacent to the locus of an occlusion, and thereafter,
deploying said collapsible cage from the interior of the outer catheter, and,
optionally but preferably treating said occlusion utilizing the treatment article.

In a yet further aspect of the invention there is provided a process for the treatment of vessels, particularly occlusions in said vessels and especially particularly chronic total occlusions which process comprises the steps of:
positioning the distal end of a device which comprises an outer catheter and an inner catheter, and a guide wire slidably deployable from the interior of said inner catheter, said inter catheter having disposed on a part of its outer surface a collapsible cage, wherein the said inner catheter and cage are adapted to be slidably deployed from the interior of said outer catheter within a vessel adjacent to the locus of an occlusion, and thereafter,
deploying said collapsible cage from the interior of the outer catheter, and,
deploying said guide wire or other element associated with said guide wire into or across the locus of an occlusion in a vessel in order to treat the occlusion,
collapsing said collapsible cage by slidably deploying said inner catheter into said outer catheter and, removing the inner catheter and outer catheter from the vessel, while retaining the guide wire and/or sleeve within the vessel.

More specifically, the device according to the invention may be used in a process for the treatment of vessels, particularly occlusions in said the vessels as pointed out above in a particularly convenient and effective manner. First, the distal end of a device according to the invention is provided to a patient in need of treatment of an occluded vessel by insertion of the distal end of the device into said vessel, and moved such that the distal end of the device is proximate to, or abutting the locus of the occlusion in a vessel, such as an artery. Once in such a position, the outer catheter is a retracted such that the cage mounted on the inner catheter is deployed and allowed to extend whereby the cage presses against the interior walls of the vessel. Such provides both a self-centering benefit, and a stabilizing benefit relative to the inner catheter 4 and any elements which it provides, viz., a sleeve 6, guide wire 8, etc. Advantageously, the inner and outer catheters are then a fixed in this position relative to each other such as by the use of a locking means as previously described although other means which effectuate such a result may also be used. Subsequently, the sleeve present within the inner catheter is it then advanced further into the vessel until it abuts or it is adjacent to the locus of the blockage or occlusion. Preferably, the placement of the sleeve with respect to both the inner catheter and the outer catheter is such that the sleeve extends not more than about 5 mm, preferably not more than about 3 mm from the end of the inner catheter in order to retain the advantage of the self-centering benefits attendant upon the use of the device according to the invention., particularly the cage. Thereafter, a guide wire, or other end element may be used to treat the occlusion in the vessel. For example, wherein a guide wire is provided within the sleeve, the guide wire may be advanced and pushed through the occlusion. Alternately, an end element other than a guide wire may also be provided via the sleeve, and/or via the inner catheter and may be used to treat the occlusion. Once the occlusion is treated, the device according to the invention can be used to deliver and to place a treatment article such as a stent at the side of the occlusion in order to minimize the requirement of any re-treatment in the future by expanding the walls of the vessel at the site of the former occlusion. It is to be understood that the use of a sleeve is optional although, such as preferably included is an element of the device according to the invention as it facilitates the placement of a guide wire or other treatment element in the close proximity of an occlusion in a vessel.

While it is to be clearly understood that in certain embodiments, the device according of the invention does not include a control device 82 as described above, it is also to be understood that according to certain particularly preferred embodiments a control device 82 is necessarily provided. Preferably, were a control device 82 is provided, such includes a configuration according to one or more of the figures described above. The control device 82 provides at least two helpful functions. First, as is described with reference to FIGS. 10A to 10C, the interaction of the ferrule element with the exterior of the inner catheter 4, more specifically the interaction of the ferrule with frustoconical segments 98 present along the length of at least a portion of the sleeve 6 can be conveniently controlled in order to provide controlled motion of the sleeve 6 relative to the inner catheter 4 and/or outer catheter 2. Additionally, the interaction of the frustoconical segments of the sleeve 6 and the flexible disk 85 help maintain the relative positioning of the sleeve 6 relative to the inner catheter 4 and/or outer catheter 6. Second, the control device 82, particularly in the embodiments described above with reference to one or more of the figures are easily controllable by using a single hand. Such may be convenient to a surgeon in an operating theater, or other health professional in an appropriate setting during the treatment of a patient.

The above treatment processes may be performed by a human operator, e.g., a surgeon or other health care provider, or by a computer-controlled device such as by a machine or robotic device which is optionally controlled by a human operator, e.g., a surgeon or other health care provider.

The device according to the invention can be fabricated from any suitable material, and as preferably fabricated from one or more materials which can be sterilized prior to use. By way of nonlimiting example, the cage according to the invention can be made from any flexible material, preferably a flexible material which has a “memory” namely in that, subsequent to compression, twisting, and like when unhindered from such compressive or twisting forces returns to, or seeks to return to, its original configuration. For example, metals, polymers, and especially sheep-memory metal alloys such as Nitinol® or Elgiloy® coming to favorable consideration. By way of nonlimiting example, the remaining non-metallic portions of the device according to the invention can be made from any of a number of medical grade thermoplastic polymers which can be configured to provide the final shape required of a relevant portions of the device. For example, one of more of the catheters ends/or the sleeve can be manufactured from FEP, PFA, polyurethane, nylon, polyolefins such as polyethylene or polypropylene, polyvinylchloride, polyesters, Pebax®, ABS, PEEK, polyacetates, polyacrylates, polysulfones, blends or one or more thereof, and the like. Such polymers can be amenable to sterilization such as by immersion in cold sterile lint compositions or solutions. By way of nonlimiting example, the remaining metallic portions of the device can be produced from any suitable medical grade metal and or metal alloys such as for example stainless steel, as well as nickel titanium alloys.

The device according to the invention, whether in whole or only parts thereof, can be provided as a single-type use device wherein part or all of the device are provided in a package and are intended to be used in only a single medical procedure, and thereafter discarded and not reused. Alternately, the device according to the invention, whether in whole or only parts thereof can be provided as a multi-type use device where in one or more parts thereof are provided and are intended to be sterilized or otherwise treated, and reused at least once, preferably are intended to be reused a number of times prefer example, it is contemplated that the control device may be configured so to be detachable from the remaining elements of a device according to the invention and that the control device may be reused a number of times whereas the remaining elements of the device according to the invention are intended to be used a single time for the treatment of only a single patient, and thereafter discarded. The one or more elements making up the device according to the invention can be also be provided as a kit to wherein any surgeon, physician, or other healthcare professional is required to assemble one or more parts of the device according to the invention in part to its use. It is also contemplated that the device according to the invention may be provided in a kit to wherein one or more parts thereof are provided in multiple amounts, such as for example providing a single control device by providing a multiplicity of one or more of inner catheter, outer catheter, sleeve, guide wire, etc. it is of course also clearly contemplated that a device according into the invention can be provided, fully assembled and ready to use in a sterile packaging and require no assembly prior to its use in the treatment of a patient.

Although this invention has been shown and described with respect to the detailed embodiments thereof, it will be understood by those of skill in the art that various changes may be made and equivalents may be substituted for elements thereof without departing from the scope of the invention. In addition, modifications may be made to adapt a particular situation or material to the teachings of the invention without departing from the essential scope thereof. Therefore, it is intended that the invention not be limited to the particular embodiments, disclosed in the above detailed description, but that the invention will include all embodiments falling within the scope of the appended claims.

1. (canceled)

2. (canceled)

3. (canceled)

4. (canceled)

5. (canceled)

6. (canceled)

7. (canceled)

8. (canceled)

9. A device adapted for the treatment of occlusions, said device comprising: a sleeve, an outer catheter and an inner catheter, said inner catheter having disposed on a part of its outer surface a collapsible cage, wherein the said inner catheter and cage are adapted to be slidably deployed from the interior of said outer catheter; and, a control device at or proximate to the end of the device which comprises a seating collar having extending outwardly a pair of control grips, each control grip having a connecting end depending from the seating collar, and a terminal end at the opposite end thereof, a linkage extending from a portion of the control grip at a point proximate to the terminal end therefrom and having another end from which depends a split ferrule.

10. A device according to claim 9, wherein the inner walls of the ferrules are profiled having a reverse frustoconical segments, which engage a part of the sleeve when the pair of control grips are compressed.

11. A device according to claim 9, wherein the device further comprises a guide wire within the inner catheter.

12. A device according to claim 9, wherein the device comprises a treatment article selected from: a stent, a balloon tipped catheter, a dottering auger, a laser catheter, and an atherectomy catheter.

13. A device according to claim 9, wherein the collapsible cage comprises three legs.

14. A device according to claim 9, wherein the collapsible cage comprises four legs.

15. A process for the treatment of occlusions, in a blood vessel of a human subject which process comprises the steps of:
positioning the distal end of a device according to claim 9 within the blood vessel of a human subject adjacent to the locus of an occlusion, and thereafter,
deploying said collapsible cage from the interior of the outer catheter, and,
optionally but preferably treating said occlusion utilizing the treatment article.

16. A process for the treatment of occlusions, in a blood vessel of a human subject according to claim 15 which process comprises the steps of:
positioning the distal end of a the device within the blood vessel of a human subject adjacent to the locus of an occlusion, and thereafter,
deploying said collapsible cage from the interior of the outer catheter, and,
deploying said guide wire or other element associated with said guide wire into or across the locus of an occlusion in the blood vessel in order to treat the occlusion,
collapsing said collapsible cage by slidably deploying said inner catheter into said outer catheter and, removing the inner catheter and outer catheter from the blood vessel, while retaining the guide wire within the vessel.
